(19) United States
(12) Patent Application Publication
(10) Pub. No.: US 2023/0178225 A1
(43) Pub. Date: Jun. 8, 2023

(54) AUTO-DETECTION OF UTILIZATION PATTERNS OF RADIOLOGIST STATE LICENSES AND FACILITY PRIVILEGES IN TELE-RADIOLOGY OPERATIONS

(71) Applicant: KONINKLIJKE PHILIPS N.V., Eindhoven (NL)

(72) Inventors: Ekin KOKER, CAMBRIDGE, MA (US); Ranjith Naveen TELLIS, TEWKSBURY, MA (US); Siva Chaitanya CHADUVULA, MALDEN, MA (US); Sandeep Madhukar DALAL, WINCHESTER, MA (US); Yuechen QIAN, LEXINGTON, MA (US); Thusitha Dananjaya De Silva MABOTUWANA, REDMOND, WA (US); Jesse WAKLEY, CAMBRIDGE, MA (US)

(21) Appl. No.: 17/994,467

(22) Filed: Nov. 28, 2022

Related U.S. Application Data
(60) Provisional application No. 63/285,510, filed on Dec. 3, 2021.

(51) Int. Cl. G16H 40/20; G06Q 10/0639

(52) U.S. Cl. CPC G16H 40/20; G06Q 10/06393

(57) ABSTRACT

A non-transitory computer readable medium (26, 30) stores instructions executable by at least one electronic processor (20, 32) to a method (100) of monitoring performance of medical professionals. The method includes tracking a quantification of examinations performed by one or more medical professionals; tracking credentials under which the examinations are performed; and outputting, on at least one display device, a representation of usage of one or more credentials by one or more medical professionals of the plurality of medical professionals based on the tracked quantities of examinations and the tracked credentials under which the examinations are performed.

### CROSS REFERENCE TO RELATED APPLICATIONS

This application claims the benefit of U.S. Provisional Pat. Application Number 63/285,510 filed Dec. 3, 2021. This application is hereby incorporated by reference herein.

### FIELD

The following relates generally to the radiology arts, radiology scheduling arts, telemedicine arts, radiology examination reading arts, and related arts

### BACKGROUND

Requirements of state medical boards and quality/assurance departments of hospitals or facilities dictate that radiologists must be licensed to read studies in a state or country and be privileged to read studies from that particular hospital or that facility. This is particularly relevant in a tele-radiology context where the radiologists read studies from many hospitals or facilities that are located in various states or countries as opposed to radiologists that are employed by a single hospital or a facility that read studies only from that hospital or facility.

There are costs associated with licensing the radiologists in a state or country and privileging them for a hospital or a facility. For example, the licenses and privileges are renewed annually or every two years and there are fees associated with the renewals. Therefore, licensing and privileging is an ongoing operational cost for a tele-radiology company that employs many radiologists who serve various hospitals or facilities that are located in different states or countries. Thus, it is important to identify how well state licenses and hospital/facility privileges are being utilized to improve efficiency of tele-radiology operations.

Tele-radiology companies provide a platform on which many (e.g., hundreds) of radiologists offer their services to different hospitals and facilities located across, for example, the United States. In this approach, the imaging studies are performed at local hospitals to acquire patient images, which are then sent via the Internet to the tele-radiology server from which radiologists employed by the tele-radiology company retrieve the images and perform readings on the images and prepare and send back radiology reports. In performing each reading, the radiologist is engaged in a medical activity for the sourcing hospital, which requires an appropriate medical license in the state where the hospital resides as well as hospital privileges at that hospital. The tele-radiology company may perform readings for hundreds or thousands of hospitals distributed across multiple states or countries. Hence, a major contributor of operational costs for these companies are the fees for license and privilege renewals.

Existing methods of manually tracking the status of these licenses and privileges is repetitive, laborious, and prone to human errors. In addition, the timeline of license/privilege renewals of different radiologists could also be asynchronous with each other due to joining the tele-radiology group at different times and acquiring licenses or privileges for different states or hospitals/facilities at different times, which makes it harder to keep track of when to look into license or privilege renewal.

The following discloses certain improvements to overcome these problems and others.

### SUMMARY

In some embodiments disclosed herein, a non-transitory computer readable medium stores instructions executable by at least one electronic processor to a method of monitoring performance of medical professionals. The method includes tracking a quantification of examinations performed by one or more medical professionals; tracking credentials under which the examinations are performed; and outputting, on at least one display device, a representation of usage of one or more credentials by one or more medical professionals of the plurality of medical professionals based on the tracked quantities of examinations and the tracked credentials under which the examinations are performed.

In some embodiments disclosed herein, a non-transitory computer readable medium stores instructions executable by at least one electronic processor to a method of monitoring performance of medical professionals. The method includes tracking a quantification of examinations performed by one or more medical professionals; tracking credentials under which the examinations are performed, the credentials including a medical license for each medical professional and a medical facility privilege for each medical professional; and outputting, on at least one display device, a representation of usage of one or more credentials by the one or more medical professionals based on the tracked quantities of examinations and the tracked credentials under which the examinations are performed.

In some embodiments disclosed herein, a method of monitoring performance of radiologists performing radiology examinations includes tracking a quantification of examinations performed by one or more medical professionals and credentials under which the examinations are performed; and outputting, on at least one display device, a representation of usage of one or more credentials by the one or more medical professionals based on the tracked quantities of examinations and the tracked credentials under which the examinations are performed.

One advantage resides in providing a tool for tailoring license and privilege renewals of medical professionals to maximize value.

Another advantage resides in providing a tool for assessing value of medical licenses and privileges held by radiologists of an organization.

Another advantage resides in optimizing costs related to license and privilege renewals of medical professionals for a medical facility.

Another advantage resides in determining causes of inefficiencies related to license and privilege renewals of medical professionals for a medical facility.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an apparatus for monitoring usage of medical licenses and facility privileges of medical professionals in accordance with the present disclosure.

FIG. 2 diagrammatically illustrates example flow chart operations performed by the apparatus of FIG. 1.

FIG. 3 shows another example of the apparatus of FIG. 1.

FIG. 4 shows an example of one suitable illustrative output generated by the apparatus of FIG. 1.

### DETAILED DESCRIPTION

In contract radiology reading services, a tele-medicine facility employs a staff of radiologists who perform remote readings of radiology examinations for hospitals or other clients. As tele-medicine facilities operate across a country or the world, its radiologists need in some countries or states, particularly in the United States, to be specifically licensed in the originating state or country of each examination, and must further have privileges at the originating hospital. In the context disclosed herein, the United States of America is used as a representative example, but the term ‘state’ may be interpreted as any geographically or relevant jurisdiction that may have its own licensing legislation or regulation, such as a country, a province, a state, a ‘bundesland’, a union, etc. This creates a complex environment. Similar institutions often maintain a Credentialing, Licensing, and Privileging (CLP) department whose sole job is to keep its staff radiologists up to date in their licenses and privileges. This work amounts to a substantial portion of the tele-medicine facility’s costs of doing business.

The following discloses a system to track examinations performed by individual radiologists in terms of examination volume and/or relative value unit (RVU) values. For each exam, the corresponding license and hospital privilege under which the exam was done is also tracked.

These data are compiled to track the extent to which each radiologist is using each license and each privilege of that radiologist. The system employs configurable parameters (e.g. thresholds) to flag specific licenses/privileges of specific radiologists as being underutilized. This can be output, for example, as recommendations not to renew infrequently utilized licenses or privileges.

In some embodiments, the system takes into account workload change forecasts based on upcoming contracts anticipated to be coming online. Hence, if for example a radiologist has a significantly underutilized license in one state, but the tele-medicine facility is about to acquire a medical facility located in that state as a client, this can be taken into account to recommend renewing licenses of that state for the radiologists.

As used herein, the term “license” (and variants thereof) refers to a formal recognition by a regulatory agency or body that a person has passed all the qualifications to practice that profession in that state (as in the United States where medical professionals are regulated at the state level; or more generally in the relevant regulatory region). Typically, licensure requirements for initial licensing include some combination of education, training, and examination to demonstrate competency. Licensure requirements after initial licensure typically also involve continuing medical education, training, and, for some specialties, periodic re-examination. If a radiologist licensed in one state seeks licensure in another state, the existing license and any disciplinary records are considered as part of the licensing process in the new state. A radiologist coming onboard with a teleradiology service will usually have a license for at least one state.

As used herein, the term “privilege” (and variants thereof) refers to an act of (or authorization by) hospitals allowing a given radiologist to read images for their particular institution. The granting of privileges to a radiologist can define the scope of permitted activities the radiologist may engage at the facility (e.g., some radiologists will be privileged to do only preliminary reads). To read a given radiology examination for a given medical facility, the radiologist will typically be required to have credentials including both a current license to practice radiology in the state where the medical facility is located and also appropriate privilege at that medical facility. Other credentials may be required to provide certain medical specialties, such as an appropriate board certification.

While the illustrative examples relate to tele-radiology, more generally credentialing is challenging in the field of telemedicine generally. Medical professionals employed by a telemedicine entity will typically be required to have credentials (license and privilege) for each state and medical facility for which they provide services. The telemedicine paradigm advantageously enables efficient delivery of medical services to otherwise underserved communities, and also advantageously enables highly specialized medical professionals to practice their specialty over a large geographical area, thus maximizing utilization of the specialty. Efficiency can be increased by having a large number of medical professionals- (e.g. in the hundreds or more in some cases) employed by a telemedicine entity. However, this makes handling of licensing and privileging challenging.

Moreover, if a medical professional maintains credentials which are unused, this is an unnecessary cost for the telemedicine entity. This cost includes fees for renewal of licenses and privileges, but can also extend to costs for continuing medical education (CME) accredited in the target state, costs for renewal examinations, travel costs associated with such activities, “lost opportunity” costs incurred by medical services that cannot be provided while the medical professional takes the CME, and so forth. As an example, if a radiologist is licensed in a State A for which that radiologist performs few or no readings, then the costs of obtaining CME credits in programs accredited in State A, travel to State A to participate in those CME programs, fees associated with license renewal in State A and further fees for privileging in various medical facilities in State A, and time off to take the CME for State A, are all unnecessary costs for the telemedicine entity. Embodiments disclosed herein provide tools for valuating credentials of individual medical professionals and for identifying underutilized credentials.

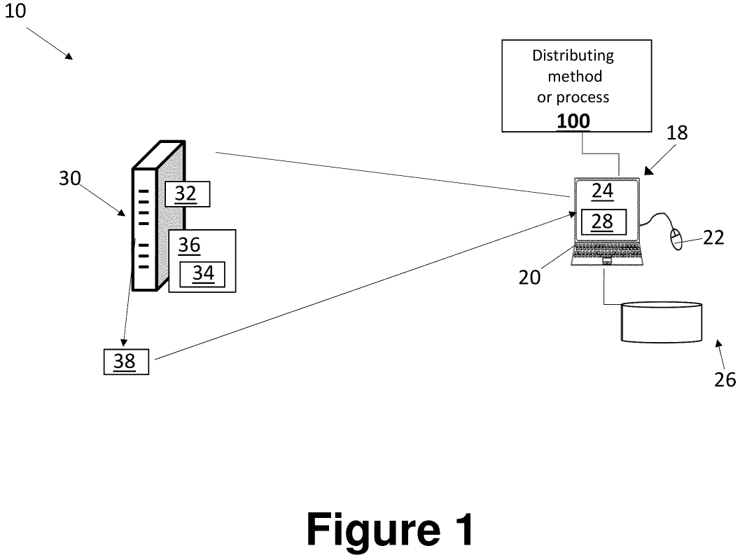

With reference to FIG. 1, an illustrative apparatus 10 for assessing utilization of credentials of medical professionals is diagrammatically shown. The apparatus 10 can comprise an electronic processing device 18 operable by the medical professionals. The electronic processing device 18 can comprise any suitable electronic device, such as a workstation computer, or more generally a computer, that is operable by a user such as a human resources (HR) department manager, a credentialing, licensing, and privileging (CLM) department manager, or so forth. The electronic processing device 18 may also include a server computer or a plurality of server computers, e.g., interconnected to form a server cluster, cloud computing resource, or so forth, to perform more complex computational tasks. The electronic processing device 18 includes typical components, such as an electronic processor 20 (e.g., a microprocessor), at least one user input device (e.g., a mouse, a keyboard, a trackball, and/or the like) 22, and a display device 24 (e.g., an LCD display, plasma display, cathode ray tube display, and/or so forth). In some embodiments, the display device 24 can be a separate component from the electronic processing device 18, or may include two or more display devices.

The electronic processor 20 is operatively connected with one or more non-transitory storage media 26. The non-transitory storage media 26 may, by way of non-limiting illustrative example, include one or more of a magnetic disk, RAID, or other magnetic storage medium; a solid-state drive, flash drive, electronically erasable read-only memory (EEROM) or other electronic memory; an optical disk or other optical storage; various combinations thereof; or so forth; and may be for example a network storage, an internal hard drive of the workstation 18, various combinations thereof, or so forth. It is to be understood that any reference to a non-transitory medium or media 26 herein is to be broadly construed as encompassing a single medium or multiple media of the same or different types. Likewise, the electronic processor 20 may be embodied as a single electronic processor or as two or more electronic processors. The non-transitory storage media 26 stores instructions executable by the at least one electronic processor 20. The instructions include instructions to generate a visualization of a graphical user interface (GUI) 28 for display on the display device 24.

The electronic processing device 18 is also in communication with a server computer 30 (shown in FIG. 1 as a server computer) that stores medical credentials 34 of a plurality of medical professionals (i.e., radiologists) in a radiologist credentials database 36 to perform medical examinations (i.e., radiology examinations). The credentials 34 can include, for example, a medical license of each medical professional, a medical facility privilege of each medical professional, board certification in a certain medical specialty, and so forth.

The server computer 34 also includes an electronic processor 32 configured to analyze the medical credentials 34 and radiology examination volume of each radiologist at each medical facility to determine which various credentials 34 should be renewed, or which credentials 34 are underutilized and for which renewal may not be justified.

The apparatus 10 is configured as described above to perform a method or process 100 of monitoring performance of medical professionals. The non-transitory storage medium 26 and/or the server computer 30 stores instructions which are readable and executable by the at least one electronic processor 20 of the electronic processing device 18 and/or the electronic processor 32 of the server computer 30 to perform disclosed operations including performing the servicing method or process 100. In some examples, the method 100 may be performed at least in part by cloud processing.

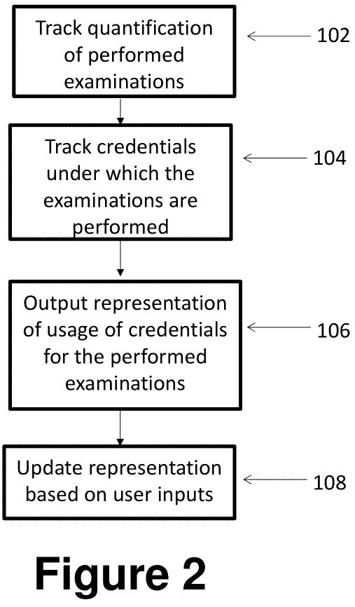

With reference to FIG. 2, and with continuing reference to FIG. 1, an illustrative embodiment of an instance of the method 100 is diagrammatically shown as a flowchart. At an operation 102, a quantification of examinations performed by a plurality of medical professionals (i.e., the radiologists) are tracked. In one example, a volume of examinations performed by the radiologists are tracked. In another example, a RVU of examinations performed by the radiologists are tracked. RVU is an approach for quantifying radiology readings which considers reading difficulty, and is specific for radiology – other metrics for quantification of examinations performed might be suitable for telemedicine professionals in other medical disciplines.

At an operation 104, credentials under which the examinations are performed are tracked. The tracked credentials can be, for example, the medical credentials 34 stored in the database 36. The operation 104 determines the credentials utilized in a given examination based on information such as an identification of the hospital or other medical facility that outsourced the radiology examination reading (from which required privilege is determined), the state within which the hospital is located (suitably obtained from an internal database listing information on serviced hospitals including address information – the state identifies the required state license), information about the reading or other medical task itself (from which any required board certification can be determined), and/or so forth.

In one contemplated variant embodiment, the operation 104 is performed at least in part each time a radiologist opens a radiology examination for reading, or each time a radiologist proceeds to file a radiology report. In such embodiments, the operation 104 may cross-check the credentials utilized in that reading against the credentials actually held by the radiologist as recorded in the credentials database 36. If this cross-check indicates the radiologist does not hold a credential required to perform the reading, then remedial action can be taken, such as issuing a warning or preventing the radiology report from being sent to the hospital. In this way, the operation 104 can optionally also serve as a check to ensure the reading radiologist has the appropriate credentials to perform each reading. In some embodiments, data can be aggregated across a teleradiology network and used to forecast staffing needs relative to current credentials being used. This information can for example be used to determine if additional resources (i.e., additional licenses or credentials) are needed, and make recommendations on how to resolve these needs. For example, the system can output a staffing forecast report on a monthly or other time basis, and/or on demand, summarizing these recommendations. This forecasting can also be taken into account when analyzing the RVU history and personal credentialing recommendations for individual radiologists. For example, the system can output an RVU history and credentials usage report for the individual radiologist that may be useful when a human resources manager or CLP department manager or other appropriate manager conducts a review meeting with the radiologist.

At an operation 106, after the operations 102 and 104 collect representative data over a statistically significant number of examinations, a representation 38 of usage of one or more credentials by one or more medical professionals of the plurality of medical professionals based on the tracked quantities of examinations and the tracked credentials under which the examinations are performed is output on the display device 24 of the electronic processing device 18. In one example embodiment, the representation 38 comprises a tree map for a specific credential 34 in which blocks of the tree map correspond to an individual radiologist, and the size of each block represents the quantification of examinations performed under that credential by the individual radiologist corresponding to that block. The user (e.g., HR or CLP manager) can readily recognize any radiologist who is underutilizing that credential, since that radiologist will be represented by a small block corresponding to a small number of examinations performed under that credential by the radiologist.

In another embodiment, the representation 38 comprises a tree map for an individual radiologist in which each block of the tree map corresponds to a credential, and the size of each block represents the quantification of examinations performed by the individual radiologist under the credential corresponding to that block. The user (e.g., HR or CLP manager) can readily recognize any credential of the radiologist that is underutilized, since that credential will be represented by a small block corresponding to a small number of examinations performed by the radiologist under that credential.

At an optional operation 108, a user operates the electronic processing device 18 to update the representation 38 displayed on the display device 24. In one example embodiment, a credential 34 of an individual radiologist is identified as underutilized if the statistics on examinations performed by the medical professional under that credential is less than a threshold. The representation 38 is updated to include an indication of the underutilized credential of the medical professional. For example, the individual grid blocks of the representation 38 corresponding to a medical professional being underutilized can color-coded.

In another example, a potential workload change forecast for the examinations based on contract data can be determined, and the representation 38 can be adjusted based on the determined potential workload change forecast.

### EXAMPLE

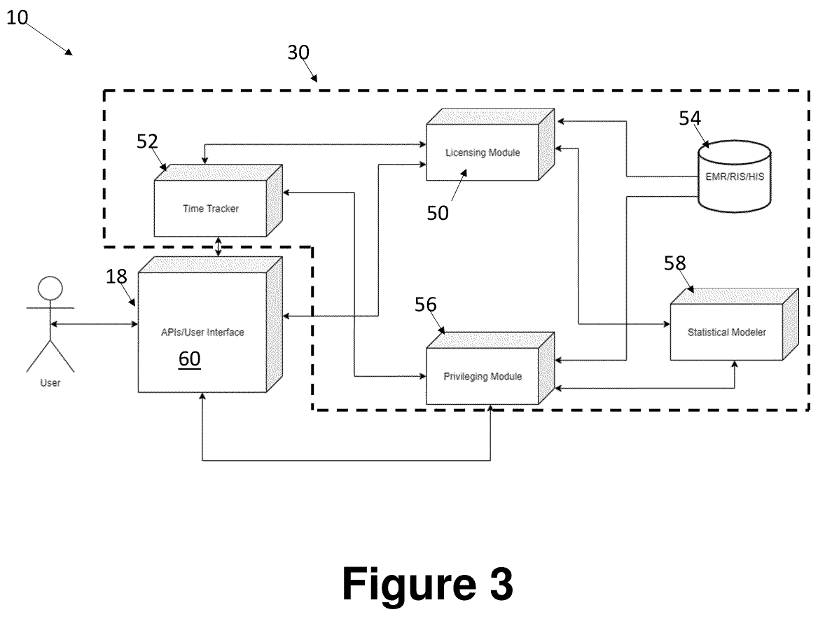

FIG. 3 shows another embodiment of the apparatus 10. As shown in FIG. 3, the server computer 30 includes one or more modules implemented in the electronic processor 32. A licensing module 50 is configured to analyze the reading volume, service level agreements (SLA) and/or RVU data for each radiologist-state pair to identify the percentage contribution of radiologist/state and state/radiologists, and determines where both are on a low efficiency end.

There can be additional variables, such as when the radiologist has joined the group, when the state license was acquired, when the state license is due for renewal, when did the radiologist read any studies from this state last time, are there enough backup radiologist in case of spike in volumes or unavailability of staff, and so forth. For example, if the radiologist has just joined, if he had just acquired the state license, the volumes could naturally be expected to be low but also expected to grow. The licensing module 50 interacts with a time-tracker module 52 to analyze of these variables and ensures the state licenses that are not supposed to be flagged as under-utilized are not flagged.

Another variable could be that on-call radiologists that are licensed in every state that read low volumes from each state only when needed should not be flagged as under-utilized either. The licensing module 50 keeps track of this information as well.

Conversely, if a radiologist has been reading a lot of volume/RVUs from a particular state for a long time or has been improving their SLAs consistently, the state license is being utilized well and it might be automatically renewed. The decision makers can also talk to the radiologist or do an analysis to understand how these efficiencies were gained (e.g. there are many facilities in that state that match the preferences/expertise of the radiologist) to apply the learnings to other radiologists as well.

The licensing module is configured to create a record for each state license (or a country-specific license in the case of international operations) by retrieving data from the available data sources in a Hospital Information System (HIS)/EMR/RIS database 54, or third party sources in the case of licensing costs. This record is then periodically updated with relevant information. For each state-radiologist pair, this data could include a name of the radiologist, a state that the radiologist is licensed, a date that state license was acquired, a date that state license is due for renewal, a volume of studies read under the license, RVUs of studies read under the license, a date that a study was read under the license last time, whether the radiologist works in an on-call basis or not, a cost of licensing, which can be found on state licensing boards’ website and provided by a third party, and so forth.

A privileging module 56 is configured to analyze the utilization patterns of hospital/facility privileges, and identify radiologist-hospital/facility pairs that might be well-utilized or under-utilized. The privileging module 56 is configured to create a record for each hospital/facility privilege by retrieving data from the available data sources in the HIS/EMR/RIS database 54, or third party sources in the case of privileging costs. This record is then periodically updated with relevant information. Some hospitals/facilities might not require privileging, and this is factored into our systems and analyses as well. For each hospital/facility-radiologist pair where privileging is required, this data could include a name of the radiologist, a hospital/facility that the radiologist is privileged for, a date that privilege was acquired, a date that privilege is due for renewal, a volume of studies read under the privilege, RVUs of studies read under the privilege, a date that a study was read under the privilege last time, whether the radiologist works in an on-call basis or not, a cost of privileging, which might come from third party sources such as credentialing office of the hospital/facility, and so forth.

A statistical modeler 58 is configured to analyses the entire reading volume and/or RVUs data to build statistical distributions for radiologist-to-state, state-to-radiologist, radiologist-to-hospital/facility, and hospital/facility-to-radiologist contributions to identify tail end of the distributions, interacting with the licensing module 50 and the privileging module 56. The statistical modeler 58 receives reading volume/RVU data from the licensing module 50 and the privileging module 56 to create statistical distributions for radiologist-to-state, radiologist-to-hospital/facility, state-to-radiologist, hospital/facility-to-radiologist contributions. This can be done in an ongoing basis, ad-hoc, or at discrete points in time (when the licenses/privileges are due). The statistical modeler 58 is configured to identify outliers and notify the licensing module 50 and the privileging module 56, which then considers additional data points (the last time a study was read using the license/privilege, on-call status for the radiologist, etc.) and notifies the user as needed. Machine-learning algorithms for detecting efficiency/inefficiency patterns will be employed in the statistical modeler 58.

The time tracker 52 is configured to analyze the timeline of all events (licensing date, privileging date, licensing due, privileging due, radiologist join, hospital/facility join, last read of radiologist from a state, last read of a radiologist from a hospital/facility, etc.) and interacts with the licensing module 50 and the privileging module 56 to provide timing information as needed. It also interacts with the user interface to notify the user when an important event is coming up (state license due for renewal, hospital/facility privilege due for renewal, etc.). These notices can come in the form of an email, message on the screen, text messages, etc. The time tracker 52 receives time data from the licensing module 50 and the privileging module 56 to create a timeline of important events. The time tracker 52 is configured to notify the licensing module 50 and the privileging module 56 when an important event is coming up (e.g. license/privilege due), which then considers additional data points (whether the license/privilege due is under-utilized or not, the last time a study was read using the license/privilege, on-call status for radiologist, etc.) and notifies the user as needed.

An application programming interface (API) or user interface (UI) 60 is implemented on the GUI 28 on the display device 24 of the electronic processing device 18 operable by a user. The API 60 is configured to allow the user to interact with the server computer 30. For example, the user can ask the server computer 30 to show them the utilization patterns of state licenses and hospital/facility privileges at a certain point in time. The server computer 30 then provides the user with a list of potential licenses and privileges that are being utilized well or under-utilized. This can also allow the user to do a root-cause analysis and find out where the efficiency/inefficiencies are coming from. The user can also check the impact of assigning/un-assigning a license or privilege on the SLAs and the system can make recommendations and display the impact of a particular license or privilege assignment so that the user can make an informed decision. Finally, the API 60 also interacts with the time tracker 52 and notifies the user before important events (state license due for renewal, hospital/facility privilege due for renewal, etc.). These notices can come in the form of an email, message on the screen, text messages, etc.

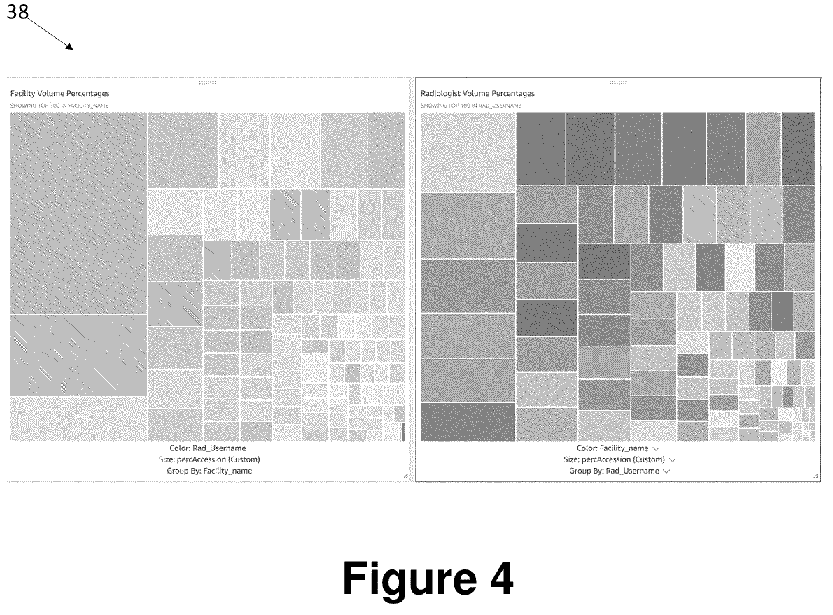

FIG. 4 shows an example of the representation 38. The representation 38 can be, for example, a tree map, and the top left and bottom right corners of the tree maps shown in FIG. 4 are the highest and lowest contributors/outliers. In order to find the well-utilized and under-utilized facility privileges, the user can tune the hyper-parameters of the apparatus 10 when it is first being set up (e.g. percentage contribution thresholds for facilities and radiologists).

Machine learning algorithms in the statistical modeler 58 can then use these thresholds and other parameters (e.g. whether the radiologist/facilities are new or not, whether the radiologist works in an on-call basis, the last time the radiologist has read any studies from that facility, the due date for the privilege, etc.) to identify the well-utilized and under-utilized privileges automatically on an on-going basis. The machine learning algorithms can use structured ways of analyzing the data to identify the outliers (e.g. a weighted average approach where the weights of each parameter is decided and re-adjusted automatically based on feedback from the user). The user will then be provided, in a timely fashion, with a ranked list of over-utilized, well-utilized and under-utilized facility privileges shown in Table 1 below.

TABLE 1

| | | | | | | | |
|---|---|---|---|---|---|---|---|
| Facility | Rad | State | Volume | % Total | % Facility | % Rad | Last Read |
| Hospital A | Rad A | MA | 30000 | 1.4% | 12% | 24% | 1 day |
| Hospital B | Rad B | NH | 19000 | 0.9% | 8% | 27% | 1 week |
| ..... | | | | | | | |
| ...... | | | | | | | |

The user can then review this list with radiologists/facility managers to decide if the privilege needs to be renewed or not. They can also pull up the advanced visualizations such as the representation 38 to dig more in to the data if needed. The API 60 will provide both the lists and the visualizations.

In some embodiments, the apparatus 10 provides an automated way of identifying utilization patterns of facility privileges. It works similarly for utilization patterns of state licenses; the only change is the user will be provided with a list of radiologist-state combinations instead of radiologist-facility combinations.

In other embodiments, when new radiologists are added, the apparatus 10 can make recommendations for states/facilities a radiologist to be licensed/privileged based on the reading volumes or the SLAs. For example, if a radiologist has experience reading certain type of studies (e.g. MR Lumbar Spine exams, CT Abdomen and Pelvis exams, etc.) or prefers to work certain hours of the day and a particular facility’s reading volumes are low or SLAs are suffering due to a lack of radiologist that can read those types of exams or can work the particular hours that a facility needs, then the apparatus 10 can recommend that radiologist to be licensed for the state that the facility is in and be privileged for that facility.

In other embodiments, when new facilities are added, the apparatus 10 can determine , if a particular radiologist’s license for a state is being under-utilized, and if the upcoming new facilities are not in that state, then it might be okay to revoke that license, which the system can alert the user about. However, if the facilities are in that state, the under-utilized license could be marked as potentially useable for those facilities and privileging module could be flagged to initiate the process for creating new privileges for that radiologist for those facilities so that their license can be utilized better.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing instructions executable by at least one electronic processor to a method of monitoring performance of medical professionals, the method including:
tracking a quantification of examinations performed by one or more medical professionals;
tracking credentials under which the examinations are performed; and
outputting, on at least one display device, a representation of usage of one or more credentials by one or more medical professionals of the plurality of medical professionals based on the tracked quantities of examinations and the tracked credentials under which the examinations are performed.

2. The non-transitory computer readable medium of claim 1, wherein the representation comprises a tree map for a specific credential in which each block of the tree map corresponds to a medical professional of the one or more medical professionals and the size of each block represents the quantification of examinations performed under that credential by the medical professional corresponding to that block.

3. The non-transitory computer readable medium of claim 1, herein the representation comprises a tree map for a specific medical professional of the one or more medical professionals in which each block of the tree map corresponds to a credential and the size of each block represents the quantification of examinations performed by the medical professional under the credential corresponding to that block.

4. The non-transitory computer readable medium of claim 1, wherein the tracked quantification of examinations performed by one or more medical professionals includes:
tracking a volume of examinations performed by the one or more medical professionals.

5. The non-transitory computer readable medium of claim 1, wherein the tracked quantification of examinations performed by one or more medical professionals includes:
tracking a relative value unit (RVU) of examinations performed by the one or more medical professionals.

6. The non-transitory computer readable medium of claim 1, wherein the method further includes:
identifying a credential of a medical professional of the one or more medical professionals as underutilized if the statistics on examinations performed by the medical professional under that credential is less than a threshold; and
including, in the representation, an indication of the underutilized credential of the medical professional.

7. The non-transitory computer readable medium of claim 6, wherein the credential comprises a medical license.

8. The non-transitory computer readable medium of claim 6, wherein the credential comprises a medical facility privilege.

9. The non-transitory computer readable medium of claim 6, wherein the indication of the underutilized credential of the medical professional includes:
color-coding the individual grid blocks corresponding to a medical professional being underutilized.

10. The non-transitory computer readable medium of claim 1, wherein the method further includes:
determining a potential workload change forecast for the examinations based on contract data; and
adjusting the representation based on the determined potential workload change forecast.

11. The non-transitory computer readable medium of claim 1, wherein the method further includes:
aggregating data from a teleradiology network to forecast usage needs relative to current credentials being used by an individual medical professional and a previous usage of the individual medical professional’s credentials; and
determine whether additional credentials for individual medical professionals are needed based on the aggregated data.

12. A non-transitory computer readable medium storing instructions executable by at least one electronic processor to a method of monitoring performance of medical professionals, the method including:
tracking a quantification of examinations performed by one or more medical professionals;
tracking credentials under which the examinations are performed, the credentials including a medical license for each medical professional and a medical facility privilege for each medical professional; and
outputting, on at least one display device, a representation of usage of one or more credentials by the one or more medical professionals based on the tracked quantities of examinations and the tracked credentials under which the examinations are performed.

13. The non-transitory computer readable medium of claim 12, wherein the representation comprises a tree map for a specific credential in which each block of the tree map corresponds to a medical professional of the one or more medical professionals and the size of each block represents the quantification of examinations performed under that credential by the medical professional corresponding to that block.

14. The non-transitory computer readable medium of claim 12, herein the representation comprises a tree map for a specific medical professional of the one or more medical professionals in which each block of the tree map corresponds to a credential and the size of each block represents the quantification of examinations performed by the medical professional under the credential corresponding to that block.

15. The non-transitory computer readable medium of claim 12, wherein the tracked quantification of examinations performed by one or more medical professionals includes:
tracking a volume of examinations performed by the one or more medical professionals.

16. The non-transitory computer readable medium of claim 12, wherein the tracked quantification of examinations performed by one or more medical professionals includes:
tracking a relative value unit (RVU) of examinations performed by the one or more medical professionals.

17. The non-transitory computer readable medium of claim 12, wherein the method further includes:
identifying a medical license credential and a medical facility privilege credential of a medical professional of the one or more medical professionals as underutilized if the statistics on examinations performed by the medical professional under that credential is less than a threshold; and
including, in the representation, an indication of the underutilized credential of the medical professional.

18. The non-transitory computer readable medium of claim 12, wherein the method further includes:
determining a potential workload change forecast for the examinations based on contract data; and
adjusting the representation based on the determined potential workload change forecast.

19. The non-transitory computer readable medium of claim 12, wherein the method further includes:
aggregating data from a teleradiology network to forecast usage needs relative to current credentials being used by an individual medical professional and a previous usage of the individual medical professional’s credentials; and
determine whether additional credentials for individual medical professionals are needed based on the aggregated data.

20. A method of monitoring performance of radiologists performing radiology examinations, the method including:
tracking a quantification of examinations performed by one or more medical professionals and credentials under which the examinations are performed; and
outputting, on at least one display device, a representation of usage of one or more credentials by the one or more medical professionals based on the tracked quantities of examinations and the tracked credentials under which the examinations are performed.
